(19) United States
(12) Patent Application Publication
(10) Pub. No.: US 2025/0032353 A1
(43) Pub. Date: Jan. 30, 2025

(54) A VIBRATOR COOLING APPARATUS AND ASSEMBLY THEREOF

(71) Applicants: Kavya Saxena, Gwalior (IN); Kuldeep Saxena, Gwalior (IN); Deepali Saxena, Gwalior (IN)

(72) Inventors: Kavya Saxena, Gwalior (IN); Kuldeep Saxena, Gwalior (IN); Deepali Saxena, Gwalior (IN)

(21) Appl. No.: 18/278,179

(22) PCT Filed: Mar. 24, 2023
(86) PCT No.: PCT/IN2023/050282

§ 371 Date: Aug. 22, 2023

(30) Foreign Application Priority Data
Feb. 6, 2023 (IN) 202321007369
Mar. 24, 2023 (WO) PCT/IN2023/050282

(51) Int. Cl. A61H 23/02; A61F 7/10

(52) U.S. Cl. CPC A61H 23/0254; A61F 7/10; A61H 2201/0153; A61H 2201/0214; A61H 2201/5025

(57) ABSTRACT

A vibration cooling apparatus and assembly is disclosed. The vibration cooling apparatus and assembly includes a housing member, a chamber, a pair of magnets connecting the chamber and the housing member. A switch is disposed on the housing member and adapted to be operated for sending a controlling instruction to the vibration cooling apparatus. The housing member comprising a power source adjacent to the switch and a motor adapted to be connected with the power source. The motor is adapted to induce vibration to the chamber upon receiving the controlling instruction from the switch.

### FIELD OF THE INVENTION

The present disclosure relates to a hand-held tool and more particularly, relates to a vibrator cooling apparatus and assembly.

### BACKGROUND

Pain may be defined as unpleasant sensory and emotional experience associated with definite or potential bodily injury due to involuntary movement during invasive process or described in respect to such damage. As in medical treatment, injections and their use are very prevalent and necessary. But the injections at the same time may cause discomfort to the patient such as pain during pricking of the needle, prevalent dimensions of fear from the needle. In many cases, needle phobia is contemplated amongst the foremost fear inducing and painful procedures in medical treatment. Pain during administering of the injection is most predominant dimensions of fear of giving injections. According to several clinical psychologists' needle fear is not limited to children but may also afflict adults. Avoidance of needle related injury such as cut, bruise is the key thing that a person with needle fear tends to do and that can be very unhealthy for people who really need a procedure involving injection.

Thus, pain and needle phobia are considered to be the most important problems that need to be monitored by some healthcare professionals.

Several existing arts may disclose procedures effective in pain management and the use of pharmacological and non-pharmacological methods to ensure sufficient optimization significantly alleviating pain during receiving of the injection or while undergoing invasive process.

All methods used for pain management without the use of medication are defined as non-pharmacological methods. Non-pharmacological approaches constitute the basis for pain management. Further, techniques such as verbal reassurance and hand-holding are helpful to calm and distract the subject but may not reduce the perceived injection pain.

Furthermore, the application of topical analgesics such as ice packs, anesthetic ointments, and/or coolant sprays has been attempted to reduce discomfort of the subject. These modes may be used with limited success due to several disadvantages such as cumbersome application, increased treatment time, and associated risks, including contact dermatitis and hyperpigmentation or hypopigmentation.

In one such existing technique, vibration may be applied to the patient receiving injection. Vibration therapy may prove to be effective in decreasing pain perception of the subject receiving the injection.

Vibration anesthesia has repeatedly been shown to effectively and safely alleviate pain sensation, most likely by reducing pain transmission from peripheral receptors to the brain. The mechanism of action for vibration anesthesia is explained in part by the gate control principle. Gate control theory may suggest that pain sensation may be dampened by co-stimulation of nerve fibers transmitting non-noxious stimuli such as vibration. Thus, applying vibration while giving injection to the subject may prove beneficial. Particularly, during cosmetic surgery, hair transplant applying vibration to the subject may be effective in reducing pain.

However, during cosmetic surgery, hair transplant the healthcare professional may choose to give anesthetic injections to the subject through a short 30 Gauge needle at a constant slow speed at multiple sites. Anesthetic injections are usually given to numb the sensations before the invasive process. Now, the healthcare professional may decide to operate a vibration device for applying vibration to the subject while giving such anesthetic injections.

In some existing problems, it may become cumbersome for the healthcare professional to operate the vibration device and simultaneously administer anesthetic injections. Thus, the healthcare professional may employ an additional healthcare professional for holding and administering either the anesthetic injections or the vibration device.

The problem aggravates further, if the healthcare professional may decide to apply a cooling agent such as ice to reduce discomfort of the subject. Now, the healthcare professionals may be reeling in to handle multiple tools, events while administering anesthetic injections to the subject. Moreover, the ice as cooling agent is an inefficient procedure which may prove to be messy and unsuitable while maintaining healthcare standards.

Thus, there is a need to develop a hand-held apparatus which may be convenient for the healthcare professionals to handle and at the same time offer the vibration and cooling anesthetic effect to the subject.

### SUMMARY

This summary is provided to introduce a selection of concepts, in a simplified format, that are further described in the detailed description of the invention. This summary is neither intended to identify key or essential inventive concepts of the invention and nor is it intended for determining the scope of the invention.

In an embodiment of the present disclosure, a vibration cooling apparatus is disclosed. The apparatus includes a housing member, at least one chamber to be detachably attached and disposed on the housing member, a switch disposed on the housing member and adapted to be operated for sending a controlling instruction to the vibration cooling apparatus. The housing member comprising a power source adjacent to the switch and at least one motor adapted to be connected with the power source. The at least one motor is adapted to induce vibration to the at least one chamber upon receiving the controlling instruction from the switch.

In an embodiment of the present disclosure, a hand-held vibration cooling assembly is disclosed. The hand-held vibration cooling assembly includes a housing member, at least one chamber to be attached and disposed on the housing member, a plurality of magnets disposed on the housing member and the at least one chamber and adapted to connect the housing member and the at least one chamber respectively. The hand-held vibration cooling assembly includes a switch disposed on the housing member and adapted to be operated for sending a controlling instruction to the vibration cooling apparatus. The housing member comprising a power source adjacent to the switch and at least one motor adapted to be connected with the power source. The at least one motor is adapted to induce vibration to the at least one chamber upon receiving the controlling instruction from the switch.

To further clarify the advantages and features of the present invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 illustrates a perspective view of a vibrator cooling apparatus, according to an embodiment of the present disclosure;

FIG. 2 illustrates another perspective view of the vibrator cooling apparatus, according to an embodiment of the present disclosure;

FIG. 3 illustrates a sectional view of a housing member of the vibrator cooling apparatus, according to an embodiment of the present disclosure; and

FIG. 4 illustrates a perspective view of a chamber of the vibrator cooling apparatus, according to an embodiment of the present disclosure.

Further, skilled artisans will appreciate that elements in the drawings are illustrated for simplicity and may not have necessarily been drawn to scale. For example, the flow charts illustrate the method in terms of the most prominent steps involved to help to improve understanding of aspects of the present invention. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having benefit of the description herein.

### DETAILED DESCRIPTION OF FIGURES

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates. Unless otherwise defined, all technical and scientific terms used herein have the same meaning as commonly understood by one of ordinary skilled in the art to which this invention belongs. The system, methods, and examples provided herein are illustrative only and not intended to be limiting.

The term “some” as used herein is defined as “none, or one, or more than one, or all.” Accordingly, the terms “none,” “one,” “more than one,” “more than one, but not all” or “all” would all fall under the definition of “some.” The term “some embodiments” may refer to one embodiment or to several embodiments or to all embodiments. Accordingly, the term “some embodiments” is defined as meaning “no embodiment, or one embodiment, or more than one embodiment, or all embodiments.”

The terminology and structure employed herein is for describing, teaching and illuminating some embodiments and their specific features and elements and does not limit, restrict or reduce the spirit and scope of the claims or their equivalents.

More specifically, any terms used herein such as but not limited to “includes,” “comprises,” “has,” “consists,” and grammatical variants thereof do NOT specify an exact limitation or restriction and certainly do NOT exclude the possible addition of one or more features or elements, unless otherwise stated, and furthermore must NOT be taken to exclude the possible removal of one or more of the listed features and elements, unless otherwise stated with the limiting language “MUST comprise” or “NEEDS TO include.”

Whether or not a certain feature or element was limited to being used only once, either way, it may still be referred to as “one or more features” or “one or more elements” or “at least one feature” or “at least one element.” Furthermore, the use of the terms “one or more” or “at least one” feature or element do NOT preclude there being none of that feature or element, unless otherwise specified by limiting language such as “there NEEDS to be one or more.” or “one or more element is REQUIRED.”

Unless otherwise defined, all terms, and especially any technical and/or scientific terms, used herein may be taken to have the same meaning as commonly understood by one having ordinary skills in the art.

Reference is made herein to some “embodiments.” It should be understood that an embodiment is an example of a possible implementation of any features and/or elements presented in the attached claims. Some embodiments have been described for the purpose of illuminating one or more of the potential ways in which the specific features and/or elements of the attached claims fulfil the requirements of uniqueness, utility, and non-obviousness.

Use of the phrases and/or terms such as but not limited to “a first embodiment,” “a further embodiment,” “an alternate embodiment,” “one embodiment,” “an embodiment,” “multiple embodiments,” “some embodiments,” “other embodiments,” “further embodiment”, “furthermore embodiment”, “additional embodiment” or variants thereof do NOT necessarily refer to the same embodiments. Unless otherwise specified, one or more particular features and/or elements described in connection with one or more embodiments may be found in one embodiment, or may be found in more than one embodiment, or may be found in all embodiments. Although one or more features and/or elements may be described herein in the context of only a single embodiment, or alternatively in the context of more than one embodiment, or further alternatively in the context of all embodiments, the features and/or elements may instead be provided separately or in any appropriate combination or not at all. Conversely, any features and/or elements described in the context of separate embodiments may alternatively be realized as existing together in the context of a single embodiment.

Any particular and all details set forth herein are used in the context of some embodiments and therefore should NOT be necessarily taken as limiting factors to the attached claims. The attached claims and their legal equivalents can be realized in the context of embodiments other than the ones used as illustrative examples in the description below.

Embodiments of the present invention will be described below in detail with reference to the accompanying drawings.

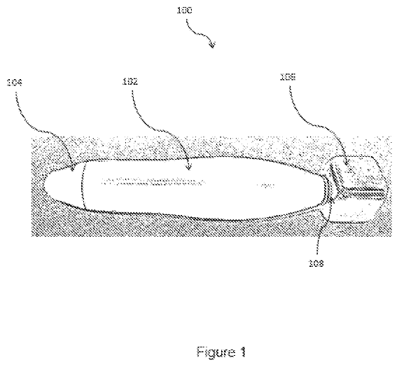

FIG. 1 illustrates a perspective view of a vibrator cooling apparatus 100, according to an embodiment of the present disclosure. In an embodiment, the vibrator cooling apparatus 100 may interchangeably be referred to as the hand-held vibration cooling assembly 100 or apparatus 100, without departing from the scope of the present disclosure. A medical professional may use the apparatus 100 during administration of an injection to a subject. The apparatus 100 may be in contact with the subject while the injection is administered. Thus, the apparatus 100 may provide a masking for pain induced in the subject during administration of the injection.

In some embodiments, the apparatus 100 may include a housing member 102, a switch 104, a chamber 106. The housing member 102 may form an elongated tubular structure housing a power source (not shown) and a motor (not shown) within the housing member 102. The housing member 102. The housing member 102 may be cylindrical in shape such that the medical professional may be able to hold the housing member 102 with ease. Further, the housing member 102 may comprise of an equivalent two halves, which may be joined or combined using fasteners. Thus, the housing member 102 may form an enclosing tubular structure housing the power source (not shown) and the motor (not shown).

In an embodiment, the switch 104 is disposed on the housing member 102. Preferably the switch 104 may be disposed at an extreme end of the housing member 102. The switch 104 may be operated by a user preferably by the medical professional operating the apparatus 100. The switch may send a controlling instruction to the apparatus 100 for initiating a vibration. Further the switch 104 may comprise of a plurality of grooves disposed on the switch such that the grooves may provide a grip to the user while operating the apparatus 100. Furthermore, the switch 104 may be made of a resilient material such that it may push button mechanism for switching ON/OFF the apparatus 100.

In a embodiment, the chamber 106 is attached and disposed on the housing member 102. Preferably, the chamber 106 may be disposed at another extreme end of the housing member 102. Said another extreme end wherein the chamber 106 is disposed is situated opposite to the switch on the housing member 102. In an example, the chamber 106 may be a cuboidal shaped container which may comprise a cooling agent. The cooling agent may be pre-filled in the chamber 106 and the chamber 106 may be sealed so that the cooling agent may not spill out. In the example, the cooling agent may be a benzene solution which may be achieve a temperature of −( )° C. upon freezing.

In some embodiment, the chamber 106 may be detachably connected to the housing member 102. In an example, the chamber 106 may be connected to the hosing member 102 using magnets. In the example, a first magnet is disposed on the another extreme end of the hosing member 102. Further, the chamber 106 may include a second magnet. The second magnet may be disposed on a face of the chamber 106. The face may be positioned to align adjacent to the another extreme end of the housing member with the first magnet. In the example, a polarity of the first magnet and the second magnet may be opposite, i.e., the first magnet may be a north pole and the second magnet may be a south pole. Thus, as the housing member 102 with the first magnet is placed in proximity to the chamber 106 with the second magnet, both the housing member 102 and the chamber 106 may be assembled due to the first magnet and the second magnet attraction forces.

Thus, the chamber 106 with the cooling agent may be detachably attached on the housing member 102. Upon application of force by the user the first magnet and the second magnet may be separated and the chamber 106 may be stored in a freezer. The freezer will provide the required environmental conditions to the chamber 106 to attain a desired freezing temperature. The user may then reassemble the chamber 106 with the housing member 102 once the freezing process is completed.

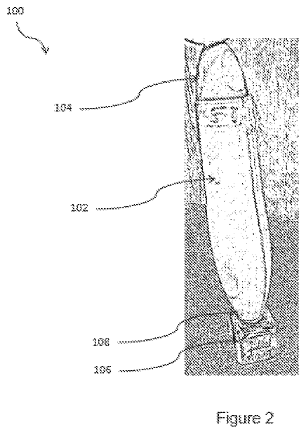

FIG. 2 illustrates another perspective view of the vibrator cooling apparatus 100, according to an embodiment of the present disclosure.

As depicted in the FIG. 2, the chamber 106 may be in contact with the subject while the medical professional administer the injection. The medical professional may hand-held the apparatus 100 gripping the housing member 102. Upon toggling the switch 104 the vibration mechanism may be initiated in the apparatus 100. Thus, the chamber 106 may receive the vibration and passes the same to the subject while staying in contact with the subject. Additionally, the chamber 106 with the cooling agent may provide a cooling sensation to the subject upon contact. Thus, the subject receives the vibration and the cooling from the apparatus 100 while being administered with the injection. The vibration and the cooling may provide advantageous benefits to the subject by masking the pain from the injection or needle pricking.

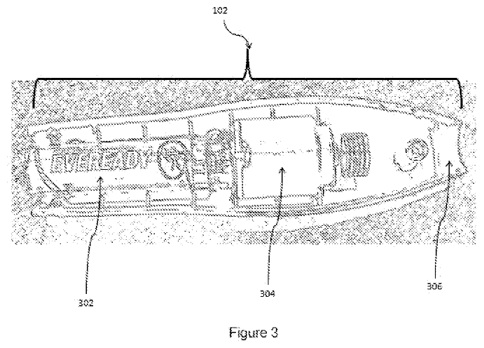

FIG. 3 illustrates a sectional view of the housing member 102 of the vibrator cooling apparatus 100, according to an embodiment of the present disclosure.

As depicted in FIG. 3, the housing member 102 is opened and only a partial sectional view of one of the halves is visible. The housing member 102 may form a cavity which includes and houses the power source 302, the motor 304 and the first magnet 306.

In an embodiment, the power source 302 may be adapted to provide operational power to the motor 304. In an example, but not limited to, the power source 302 may be a zinc-carbon battery of 1.5 Volts. In another example, the power source 302 may be disposed outside the apparatus 100 or the housing member 102 such that external power source may be applied to supply operational power to the motor 304. The switch 104 may be placed adjacent to the power source 302. Thus, the switch 104 may act by completing a circuit such that the power source 302 may supply operational power to the motor 304.

In an embodiment, a power of the motor 304 may be 20000 rounds per minute (rpm). Further, the motor 304 may be of 24 mm×27 mm dimensions and is placed inside cavity of the housing member 102. The motor 304 upon receiving operational power from the power source 304 will initiate vibrations. The vibrations may thus be induced in the apparatus 100, particularly in the chamber 106 attached to the housing member 102. The switch 104 upon receiving an input from the user may provide a controlling instruction to the motor 304 for initiating operations of the apparatus 100. The controlling instructions are indicative of supply of operational power to the motor 304 for initiating its mechanism to induce vibrations.

In an embodiment, the first magnet 306 may be disposed at the another extreme end of the housing member 102. In an example, but not limited to, the first magnet 306 may be made of a neodymium material with circular shape and a dimension of 12 mm diameter×2.5 mm thickness. In the example, the first magnet 306 may be a north pole magnet.

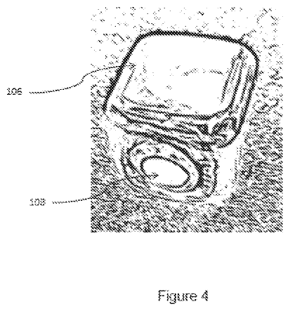

FIG. 4 illustrates a perspective view of the chamber 106 of the vibrator cooling apparatus 100, according to an embodiment of the present disclosure. The chamber 106 may include the cooling agent filled inside the chamber 106. The chamber 106 may include the second magnet 402. The second magnet 402 is disposed on the face of the chamber 106. In an example, the second magnet 402 may be made of a neodymium material with circular shape and a dimension of 12 mm diameter×2.5 mm thickness. In the example, the second magnet 402 may be a south pole magnet.

The first magnet 306 and the second magnet 402 may be attached thus detachably attaching the chamber 106 with the housing member 102.

Thus, as the medical professional operates the apparatus 100, the chamber while being attached to the housing member 102 may remain in contact with the subject. As the motor initiates operation the vibrations are induced to the subject along with the chamber acting cooling agent respectively.

In some advantages of the present invention:
1) The present invention provides an efficient means for the medical professional to administer vibration and cooling sensation to the subject. Vibration as alone technique may not work in all the cases so many of the patients require dual non-noxious stimulus that is cooling and vibration simultaneously.
2) The present invention eliminates additional manpower and provides much efficient ways to induce vibration and cooling sensation to the subject.
3) The present invention assists in masking pain and fear of needle pricks.
4) The present invention eliminates the messy procedure of applying ice which may melt during administration of injection.
5) The present invention provides a chamber with cooling agent that may last for long-duration. Thus, increasing efficiency of the procedure.
6) The present invention provides cost-effective, handy, and convenient operational usage/application of vibration and cooling properties to the subject.

While specific language has been used to describe the present subject matter, any limitations arising on account thereto, are not intended. As would be apparent to a person in the art, various working modifications may be made to the method in order to implement the inventive concept as taught herein. The drawings and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment.

1. A vibration cooling apparatus, comprising:
a housing member;
at least one chamber to be detachably attached and disposed on the housing member;
a switch disposed on the housing member and adapted to be operated for sending a controlling instruction to the vibration cooling apparatus;
wherein the housing member comprising a power source adjacent to the switch and at least one motor adapted to be connected with the power source;
wherein the at least one motor is adapted to induce vibration to the at least one chamber upon receiving the controlling instruction from the switch.

2. The vibration cooling apparatus as claimed in claim 1, wherein a first magnet is disposed on the housing member and adapted to be attached with the at least one chamber.

3. The vibration cooling apparatus as claimed in claim 2, wherein a second magnet is disposed on a face of the at least one chamber and adapted to be attached with the housing member.

4. The vibration cooling apparatus as claimed in claim 3, wherein the first magnet and the second magnet are opposite poles of neodymium material.

5. The vibration cooling apparatus as claimed in claim 4, wherein the first magnet and the second magnet are in circular shape with a 12 mm diameter×2.5 mm thickness.

6. The vibration cooling apparatus as claimed in claim 1, wherein the at least one chamber includes a cooling agent.

7. The vibration cooling apparatus as claimed in claim 1, wherein the power source is a zinc-carbon battery of 1.5 Volts.

8. The vibration cooling apparatus as claimed in claim 1, wherein the at least one motor is 20000 rpm with 24 mm×27 mm dimensions.

9. A hand-held vibration cooling assembly, comprising:
a housing member;
at least one chamber to be attached and disposed on the housing member;
a plurality of magnets disposed on the housing member and the at least one chamber, and adapted to connect the housing member and the at least one chamber respectively;
a switch disposed on the housing member and adapted to be operated for sending a controlling instruction to the vibration cooling apparatus;
wherein the housing member comprising a power source adjacent to the switch and at least one motor adapted to be connected with the power source;
wherein the at least one motor is adapted to induce vibration to the at least one chamber upon receiving the controlling instruction from the switch.
